(19) United States
(12) Patent Application Publication
(10) Pub. No.: US 2019/0029797 A1
(43) Pub. Date: Jan. 31, 2019

(54) PROSTATE REPLACEMENT DEVICE

(71) Applicants: Taresa Kenens, Woodland Park, CO (US); James Parsons, Colorado Springs, CO (US)

(72) Inventors: Taresa Kenens, Woodland Park, CO (US); James Parsons, Colorado Springs, CO (US)

(21) Appl. No.: 16/049,729

(22) Filed: Jul. 30, 2018

Related U.S. Application Data
(60) Provisional application No. 62/538,373, filed on Jul. 28, 2017.

(51) Int. Cl. A61F 2/04; A61F 2/00

(52) U.S. Cl. CPC A61F 2/04; A61F 2002/047; A61F 2/004

(57) ABSTRACT

A device for implantation into a prostate-cavity of a patient having undergone a prostatectomy procedure is disclosed herein. The device includes a bag-body having an outer-shell and an inner-volume. In a preferred embodiment, the inner-volume is filled with a saline material. The device effectively fills the void after a prostatectomy procedure, helping patients quickly recover from the current side effects of surgery and preventing incontinence without having to endure intense therapy or invasive surgery.

### CROSS REFERENCE TO RELATED APPLICATION

The present application is related to and claims priority to U.S. Provisional Patent Application No. 62/538,373 filed Jul. 28, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of prostatectomy procedures and more specifically relates to a device for implantation into a patient having undergone a prostatectomy procedure.

### 2. Description of Related Art

Prostatectomy is a medical term for the surgical removal of all or part of the prostate gland. This operation is done for benign conditions that cause urinary retention, as well as for prostate cancer and other cancers of the pelvis. There are two main types of prostatectomies: a simple prostatectomy when only part of the prostate is removed; and a radical prostatectomy when the entire prostate gland, the seminal vesicles and the vas deferens are removed. Prostatectomy patients often experience incontinence, for which they may have to endure pelvic floor therapy, which can be intense and isn't practical for the elderly; or undergo a second invasive procedure. Additionally, the embarrassment and inconveniences of incontinence may lead to a slower, prolonged recovery. An effective alternative is required.

U.S. Pub. No. 2004/0143343 5 to Joseph Grocela relates to a post-radical prostatectomy continence implant. The described post-radical prostatectomy continence implant includes a spacer useful for relieving excess pressure exerted on a sphincter muscle by an anatomical structure. A preferred embodiment relates to a urethral spacer. The urethral spacer is a disk-like element formed of an absorbable, biodegradable material, and includes a slot through a portion of the device which permits the disk to be opened up and positioned around the urethra. The urethral spacer is sutured in place around the urethra adjacent to or around the urinary sphincter, preferably after a radical prostatectomy.

### BRIEF SUMMARY OF THE INVENTION

In view of the foregoing disadvantages inherent in the known prostatectomy procedures art, the present disclosure provides a novel prostate replacement device. The general purpose of the present disclosure, which will be described subsequently in greater detail, is to provide a device which effectively fills the void after a prostatectomy procedure. This helps patients quickly recover from the current side effects of surgery and prevents incontinence without having to endure intense therapy or invasive surgery. The device may surround the urethra, much like the prostate gland does.

A device is disclosed herein. The device includes a bag-body which may be made from a first bio-compatible material. The bag-body may include an outer-shell having a groove therein forming a channel extending substantially vertical through a height of the bag-body when the device may be implanted into the prostate-cavity. In one embodiment, the channel may be sized to receive a urethra of the patient when the device may be implanted into the prostate-cavity. Further, the inner-volume may be filled with a second bio-compatible material and may be configured to provide a firmness to the bag-body.

A method of using device is also disclosed herein. The method of using device may comprise the steps of: providing the device as above; surgically implanting the bag-body into the prostate-cavity; positioning the channel over the urethra of the patient; and securely positioning the urethra within the channel.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a prostate replacement device, constructed and operative according to the teachings of the present disclosure.

FIG. 1 is a front-side perspective view of the device during an ‘in-use’ condition, according to an embodiment of the disclosure.

FIG. 2A is a front-side perspective view of the device of FIG. 1, according to an embodiment of the present disclosure.

FIG. 2B is a front-side perspective view of the device of FIG. 1, according to an embodiment of the present disclosure.

FIG. 3A is a front-side perspective view of the device of FIG. 1, according to an embodiment of the present disclosure.

FIG. 3B is a front-side perspective view of the device of FIG. 1, according to an embodiment of the present disclosure.

FIG. 4 is a flow diagram illustrating a method of use for device, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to prostatectomy procedures and more particularly to a prostate replacement device as used to improve the device for implantation into a patient having undergone a prostatectomy procedure.

Generally, disclosed is a bag formed of a biologically inert material, which may be implanted into a cavity from which a prostate gland was removed. The bag may be grooved to surround a urethra. The bag when filled with saline or an otherwise medically sound material may wrap around the urethra providing support for a bladder while at the same time filling the void that remains once the prostate has been removed. In use, the device may with the swift recovery of surgical patients and may provide an ideal substitute for the more intense Pelvic Floor Therapy Regimen to which some patients may not be able to adhere. The exact specifications may vary upon manufacturing.

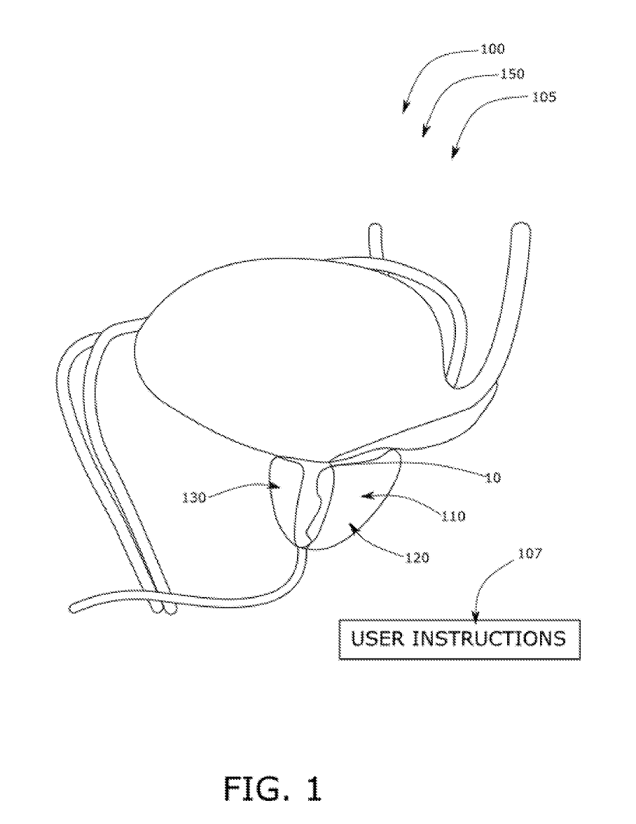

Referring now more specifically to the drawings by numerals of reference, there is shown in FIGS. 1-4, various views of a device 100.

FIG. 1 shows a device 100 during an ‘in-use’ condition 150, according to an embodiment of the present disclosure. As Illustrated, the device 100 may include a bag-body 110. The bag-body 110 may include an outer-shell 120 and an inner-volume 130. The device 100 may be used for implantation into a prostate-cavity 10 of a patient having undergone a prostatectomy procedure.

According to one embodiment, the device 100 may be arranged as a kit 105. The kit 105 may include set of user instructions 107. The instructions 107 may detail functional relationships in relation to the structure of the device 100 (such that the device 100 can be used, maintained, or the like, in a preferred manner).

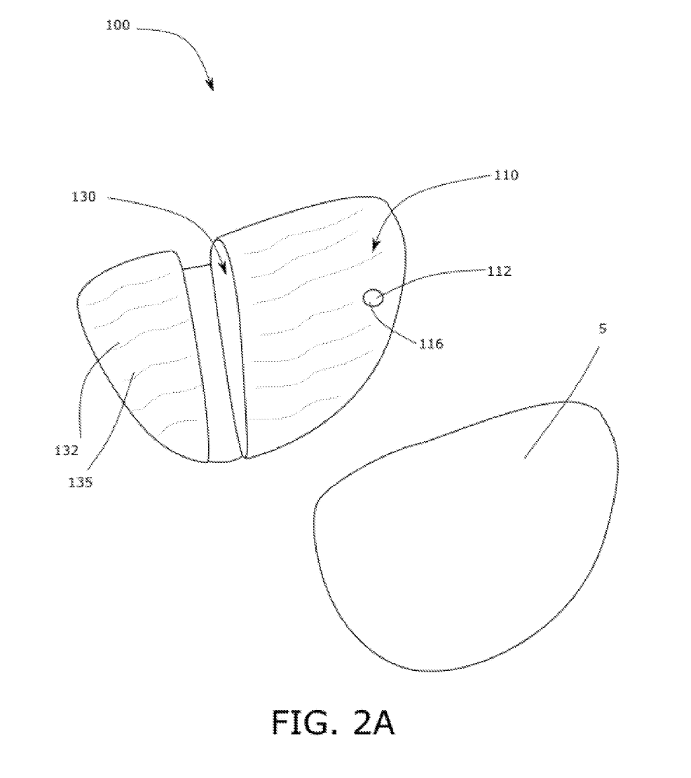

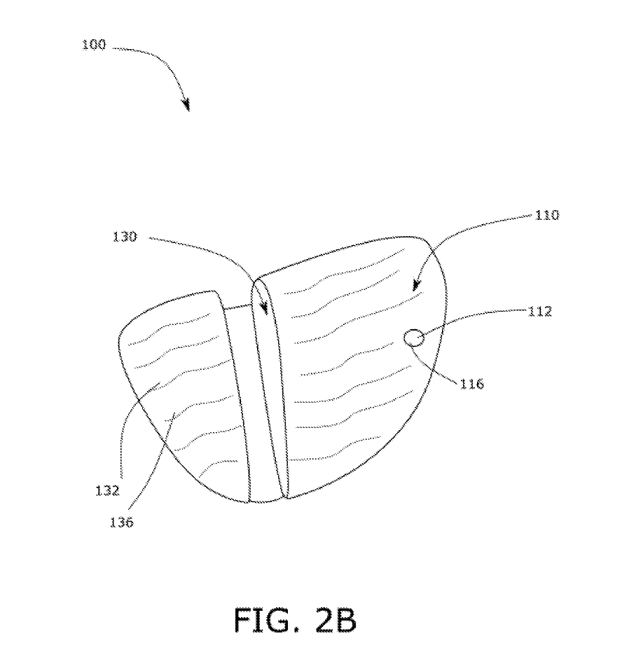

FIGS. 2A-2B show front-side perspective views of the device 100 of FIG. 1, according to an embodiment of the present disclosure. The bag-body 110 may be made from a first bio-compatible material 112. In a preferred embodiment, the first bio-compatible material 112 may comprise a first silicone material 116. In this embodiment, the first silicone material 116 may be an elastomer silicone. Preferably, the bag-body 110 may be pliable but firm to replicate a removed-prostate 5 and to prevent harm to the surrounding tissue and organs. As shown, in a preferred embodiment, the bag-body 110 may be substantially equal in size and shape to the removed-prostate 5 of the patient.

As above, the bag-body 110 may include the inner-volume 130. In the preferred embodiment, the inner-volume 130 may be filled with a second bio-compatible material 132 configured to provide a firmness to the bag-body 110, thus aiding in the replication of the removed-prostate 5. In this embodiment, the second bio-compatible material 132 may comprise a saline material 135. The saline material 135 includes a mixture of sodium chloride and sterile water to prevent growth of bacteria or microorganisms, and thus, minimizing any chance of infection.

However, other materials may be contemplated for the second bio-compatible material 132. For example, the second bio-compatible material 132 may comprise a second silicone material 136 (as shown in FIG. 2B). This second silicone material 136 may be a gel. The second silicone material 136 may provide a firmer structure to the bag-body 110.

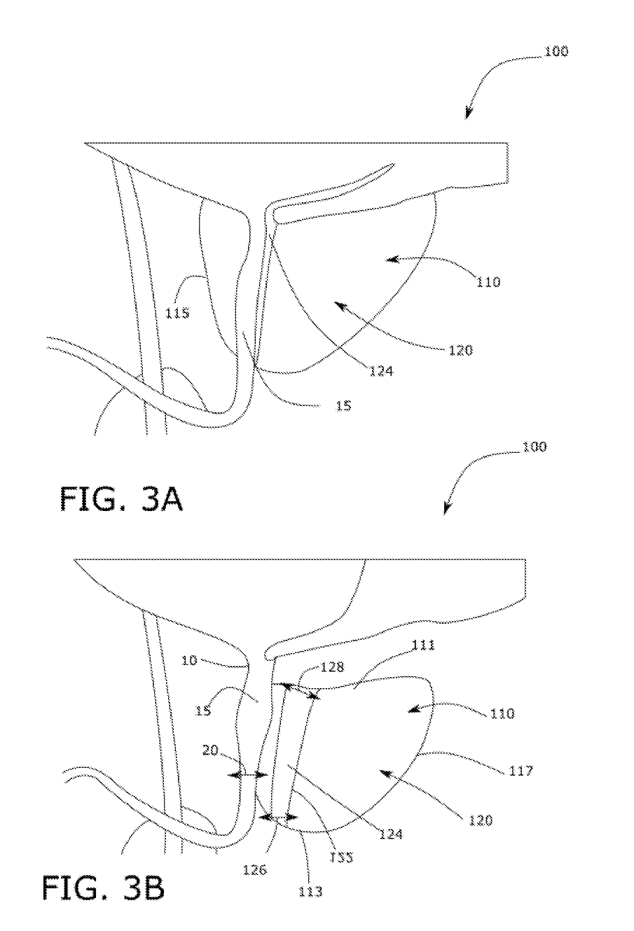

FIGS. 3A-3B show front-side perspective views of the device 100 of FIG. 1, according to an embodiment of the present disclosure. As above, the bag-body 110 may include the outer-shell 120. As shown, the outer-shell 120 may include a groove 122 therein forming a channel 124 extending substantially vertical through a height 114 of the bag-body 110 when the device 100 is implanted into the prostate-cavity 10. Further, as shown, the bag-body 110 may include a top-side 111, a bottom-side 113, a left-side 115 and a right-side 117 when implanted into the prostate-cavity 10 of the patient and in this embodiment, the channel 124 may extend vertically from the top-side 111 to the bottom-side 113 when implanted into the prostate-cavity 10 of the patient.

As shown in these figures, the channel 124 may be sized to receive a urethra 15 of the patient when the device 100 is implanted into the prostate-cavity 10. This may be particularly useful for preventing incontinence as the channel 124 is able to support the urethra 15, similarly to how the removed-prostate 5 (FIG. 2A) supported the urethra 15 pre-prostatectomy procedure.

The channel 124 may include a channel-width 126 substantially equal to a urethra-width 20 of the urethra 15. In one embodiment, the channel-width 126 may be between 7 mm-10 mm. In addition, in one embodiment, the channel 124 may include a channel-depth 128 between 7 mm-10 mm. Specifications may vary, but preferably the channel 124 includes a shape and size configured to receive and support the urethra 15.

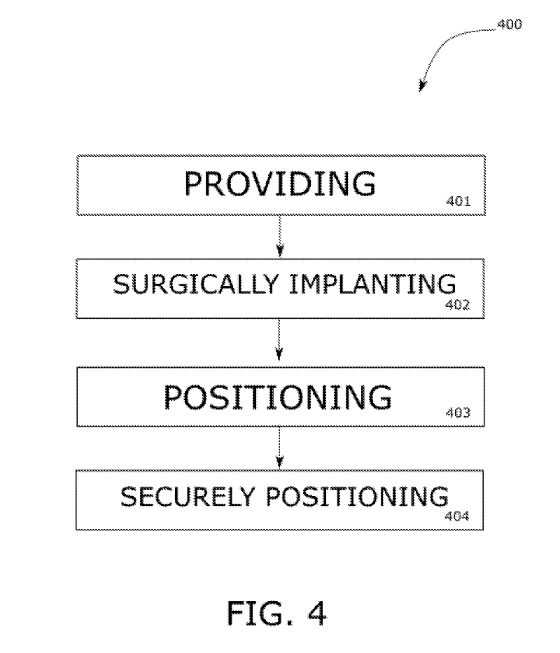

FIG. 4 is a flow diagram illustrating a method of implanting a device for implantation into a prostate-cavity of a patient having undergone a prostatectomy procedure 400, according to an embodiment of the present disclosure. As illustrated, the method of implanting a device for implantation into a prostate-cavity of a patient having undergone a prostatectomy procedure 400 may include the steps of: step one 401, providing the device 100 as above; step two 402, surgically implanting the bag-body 110 into the prostate-cavity 10; step three 403, positioning the channel 124 over the urethra 15 of the patient; and step four 404, securely positioning the urethra 15 within the channel 124. In one method of use embodiment, the device 100 may be implanted during the prostatectomy procedure, after removal of the prostate from the patient. In another method of use embodiment, the device 100 may be implanted after the prostatectomy procedure. In this example, the implantation procedure may be days or weeks after the prostatectomy procedure.

It should be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for device 100 (e.g., different step orders within above-mentioned list, elimination or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A device for implantation into a prostate-cavity of a patient having undergone a prostatectomy procedure, the device comprising:
a bag-body being made from a first bio-compatible material, the bag-body including:
an outer-shell having;
a groove therein forming a channel extending substantially vertical through a height of the bag-body when the device is implanted into the prostate-cavity, the channel being sized to receive a urethra of the patient when the device is implanted into the prostate-cavity; and
an inner-volume being filled with a second bio-compatible material, the second bio-compatible material being configured to provide a firmness to the bag-body.

2. The device of claim 1, wherein the bag-body is substantially equal in size to a removed-prostate of the patient.

3. The device of claim 2, wherein the bag-body is substantially equal in shape to the removed-prostate of the patient.

4. The device of claim 1, wherein the first bio-compatible material comprises a first silicone material.

5. The device of claim 4, wherein the first silicone material is an elastomer silicone.

6. The device of claim 1, wherein the outer-shell of the bag-body is pliable.

7. The device of claim 1, wherein the second bio-compatible material comprises a saline material.

8. The device of claim 1, wherein the second bio-compatible material comprises a second silicone material.

9. The device of claim 8, wherein the second silicone material is a gel.

10. The device of claim 1, wherein the channel includes a channel-width substantially equal to a urethra-width of the urethra.

11. The device of claim 1, wherein the channel-width is between 7 mm-10 mm.

12. The device of claim 1, wherein the channel includes a channel-depth between 7 mm-10 mm.

13. The device of claim 1, wherein the bag-body includes a top-side, a bottom-side, a left-side and a right-side when implanted into the prostate-cavity of the patient.

14. The device of claim 13, wherein the channel extends substantially vertically from the top-side to the bottom-side when implanted into the prostate-cavity of the patient.

15. A device for implantation into a prostate-cavity of a patient having undergone a prostatectomy procedure, the device comprising:
a bag-body being made from a first bio-compatible material, the bag-body including:
an outer-shell having;
a groove therein forming a channel extending substantially vertical through a height of the bag-body when the device is implanted into the prostate-cavity, the channel being sized to receive a urethra of the patient when the device is implanted into the prostate-cavity; and
an inner-volume being filled with a second bio-compatible material, the second bio-compatible material being configured to provide a firmness to the bag-body;
wherein the bag-body is substantially equal in size to a removed-prostate of the patient;
wherein the bag-body is substantially equal in shape to the removed-prostate of the patient;
wherein the first bio-compatible material comprises a silicone material;
wherein the outer-shell of the bag-body is pliable;
wherein the second bio-compatible material comprises a saline material;
wherein the channel includes a channel-width substantially equal to a urethra-width of the urethra;
wherein the bag-body includes a top-side, a bottom-side, a left-side and a right-side when implanted into the prostate-cavity of the patient; and
wherein the channel extends vertically from the top-side to the bottom-side when implanted into the prostate-cavity of the patient.

16. The device of claim 17, further comprising set of instructions; and
wherein the device is arranged as a kit.

17. A method of implanting a device for implantation into a prostate-cavity of a patient having undergone a prostatectomy procedure, the method comprising the steps of:
providing the device for implantation into the prostate-cavity of the patient having undergone a prostatectomy procedure, the device including:
a bag-body being made from a first bio-compatible material, the bag-body including:
an outer-shell having;
a groove therein forming a channel extending substantially vertical through a height of the bag-body when the device is implanted into the prostate-cavity, the channel being sized to receive a urethra of the patient when the device is implanted into the prostate-cavity; and
an inner-volume being filled with a second bio-compatible material, the second bio-compatible material being configured to provide a firmness to the bag-body;
surgically implanting the bag-body into the prostate-cavity;
positioning the channel over the urethra of the patient; and
securely positioning the urethra within the channel.

18. The method of claim 17, wherein the device is implanted during the prostatectomy procedure, after removal of a prostate of the patient.

19. The method of claim 17, wherein the device is implanted after the prostatectomy procedure.
